(19) United States
(12) Patent Application Publication
(10) Pub. No.: US 2014/0262740 A1
(43) Pub. Date: Sep. 18, 2014

(54) SEPARATION OF IMPURITIES DURING EXTRACTION PROCESSES

(71) Applicant: GTC Technology US LLC, Houston, TX (US)

(72) Inventor: Michael McCaulley, Sugar Land, TX (US)

(73) Assignee: GTC Technology US LLC, Houston, TX (US)

(21) Appl. No.: 14/213,827

(22) Filed: Mar. 14, 2014

Related U.S. Application Data
(60) Provisional application No. 61/781,420, filed on Mar. 14, 2013.

(51) Int. Cl. C07C 7/08

(52) U.S. Cl. CPC C07C 7/08
USPC 203/43; 203/71

(57) ABSTRACT

A process for the removal of sulfur compounds from a hydrocarbon stream is disclosed. The process includes extractive distillation of a feed stock coupled with a solvent recovery column having a vapor side draw containing the sulfur compound impurities.

### CROSS-REFERENCES TO RELATED APPLICATIONS

This Application claims the benefit under 35 U.S.C. §119(e) of U.S. Provisional Patent Application No. 61/781,420 filed Mar. 14, 2013 which is incorporated herein by reference in its entirety as if fully set forth herein.

### BACKGROUND OF THE INVENTION

1. Field of the Invention

The invention relates to hydrocarbon refining, and more particularly to a process for removing sulfur compounds and other impurities from hydrocarbons during an extraction process.

2. Description of the Related Art

The major source of gasoline sulfur (up to 98%) is from the gasoline produced from fluid catalytic cracking (FCC), which comprises 30 to 70% of the gasoline pool. One of the most effective ways to remove the sulfur from gasoline is to hydrotreat the FCC gasoline. However, this stream contains significant amounts of olefinic compounds, and hydrotreating these compounds substantially reduces the octane rating of the blended gasoline.

Many valuable compounds are produced by hydrocarbon extraction processes (liquid-liquid extraction and/or extractive distillation). Extraction is used on essentially all hydrocarbon molecules from methane to lube oils (Wax and aromatics removal) and beyond to produce high purity chemical products. Some of the major applications and products include, but are not limited to: extraction of carbon dioxide, hydrogen sulfide, acetylene, butadiene, isoprene, benzene, toluene and xylenes, the production of low aromatic Fuels and production of various specialty hydrocarbon products such as lube oil, aromatic and non-aromatic Solvents.

Although the extraction process technology in the petrochemical industry is a well-developed field and provides major sources of fuels and chemical products; it is not currently used to effectively remove impurities (see Table 1) from the extracted hydrocarbons. Many impurities are co-extracted with these various hydrocarbons causing major challenges to produce high purity chemicals from the extracted hydrocarbons. Distillation does not typically work for separating the co-extract impurities from the extracted products. The typical method of impurity removal from the extract hydrocarbons is hydrotreating. This is often necessary in order to separate these numerous impurities from these extracted hydrocarbons. Hydrotreating converts the impurities (sulfurs, oxygenates and nitriles) to hydrogen sulfide, water and ammonia, which readily separates from the remaining extraction products. Hydrotreating continues to be the chosen method especially for sulfur removal although its application is often problematic. Hydrotreating requires numerous steps and additional cost to provide the required impurity removal efficiencies and the chemical product purities. As such, hydrotreating often destroys the value and purity of the extracted hydrocarbons by producing byproducts. Even so, hydrotreating is the standard for removing sulfur molecules from hydrocarbons. Using current technology, sulfur compounds and most other impurities are extracted concurrently with the valuable extraction products. This causes a new set of processing and purification challenges in order to meet chemical product specifications.

There is therefore a need for a process that removes impurities from hydrocarbons during an extraction process without compromising the quality of the extracted hydrocarbons.

### SUMMARY OF THE INVENTION

The claimed invention is directed to the separation of sulfur compounds and other impurities during an extraction process such as liquid/liquid or extractive distillation, which separates and remove impurities such as sulfur compounds, nitriles and oxygenated hydrocarbons from extracted hydrocarbons comprising C₁₀ and lighter hydrocarbons during extraction processes (liquid-liquid extraction or extractive distillation). The claimed process avoids the value downgrade resulting from having these impurities (sulfur, nitrogen and oxygen compounds) present in the extracted hydrocarbons and it also avoids the additional processing of these hydrocarbons to remove these impurities, thereby eliminating significant loss of these valuable hydrocarbon products.

This invention is related to the incorporation of an extractive process into refining processes to extract sulfur compounds in the hydrocarbon streams. Particularly preferred streams for use with the invention are derived from, for example, a Coker naphtha source, a thermal steam cracked source or a fluid catalytic cracker (FCC) unit. Gasoline from a FCC unit is particularly preferred for use with the invention.

The gasoline stream may comprise single and multi-ring aromatics, single and multi-ring naphthenes, olefins, paraffins, thiophenes, benzothiophenes, sulfides, disulfides, thiols, tetrahydrothiophenes, and dihydrobenzothiophenes, having boiling points ranging from about 35° C. to about 260° C.

According to the invention, the extract stream is separated from the sulfur compounds and other impurities, which can be hydrodesulfurized with a conventional or improved HDS (hydrodesulfurization) unit. In this way, the octane rating of the desulfurized FCC gasoline can be preserved.

In an embodiment, the process according to the invention comprises an extractive distillation process comprising an extractive distillation column and a solvent recovery column having a vapor side-draw. In other embodiments of the invention, the process according to the invention is carried out using divided wall distillation, solvent stripping or through the use of dual distillation units.

### BRIEF DESCRIPTION OF THE DRAWINGS

FIG. 1 shows a currently existing process for the removal of aromatics and sulfur compounds as is known in the prior art;

FIG. 2 shows a process for the removal of impurities and sulfur compounds using a side-draw in accordance with an embodiment of the claimed invention;

FIG. 3 shows a process for the removal of impurities using divided wall distillation in accordance with an embodiment of the claimed invention;

FIG. 4 shows a process for the removal of impurities using additional solvent stripping in accordance with an embodiment of the claimed invention; and

FIG. 5 shows a process for the removal of impurities using dual distillation units in accordance with an embodiment of the claimed invention.

### DETAILED DESCRIPTION OF EXEMPLARY EMBODIMENTS

Extractive processes within the scope of the invention include extractive distillation and liquid-liquid extraction. The feedstock comprising C5 to C10 hydrocarbons is fed to an extractive process where a proper extractive solvent or mixed solvent is used to extract the sulfur compounds and aromatics into an extract stream. At the same time, olefinic, naphthenic, and paraffinic compounds in the gasoline stream are rejected by the solvent into a raffinate stream. The sulfur compounds include mainly mercaptans, sulfides, disulfides, thiophenes, benzothiophenes and dibenzothiophenes. The extract stream (with sulfur concentrates) is then fed to an HDS unit for sulfur removal.

A currently existing process for the removal of impurities as is known in the prior art in set forth in FIG. 1. In this process, a gasoline stream is subjected to an extractive distillation process to concentrate the sulfur compounds in an extract stream and reject olefins to a raffinate stream, and the extract stream is subjected to hydrodesulfurization to remove sulfur compounds. In this process, the extract stream is processed in a solvent recovery column using stripping stream, which separates the solvent (that is subsequently recycled back to the extractive distillation column) from the aromatics and sulfur compounds that are removed for further processing. During further processing, the aromatics compounds are separated from the sulfur compounds and polar impurities.

In an embodiment of the claimed invention, a process for the removal and separation of impurities including sulfur compounds during an extraction process is provided. In this process, set forth in FIG. 2, a hydrocarbon feedstock is subjected to extractive distillation to extract the sulfur compounds, impurities and aromatics into an extract stream. At the same time, non-aromatics such as olefinic, naphthenic, and paraffinic compounds in the feedstock are rejected into a raffinate stream. The extract stream comprising the aromatics, impurities and sulfur compounds is further subjected to a solvent recovery step using stripping stream. In the solvent recovery column, aromatics without impurities are separated and the sulfur compounds and impurities are concentrated and removed through a side draw. The side draw can be either a vapor side draw or a liquid side draw. Recycled solvent from the solvent recovery column is subsequently introduced into the extractive distillation column and the upper portion of the solvent recovery column.

Taking a side product during the solvent recovery operation and producing an impurity and sulfur concentrate, removes a significant amount of the sulfur and other impurities from the extracted hydrocarbon and avoids sulfur and impurity contamination of the processed hydrocarbons (both raffinate and extracted hydrocarbons), and also precludes the need for the subsequent required treatment of these extracted hydrocarbons by hydrotreating, adsorption or other onerous means, which is required to produce purity chemical and fuel products. The claimed invention thus results in a significant savings in capital investment, chemical usage (catalyst and hydrogen) and energy usage.

Solvents that are used in the claimed invention are chosen based upon whether they extrac sulfur and rejecting olefins in the FCC gasoline. Also, the boiling point of the ED solvents should be high enough to be recovered in the solvent stripper and not to contaminate the extracted products. The non-limiting solvent examples include sulfolane, 3-methylsulfolane, 2,4-dimethylsulfolane, 3-ethylsulfolane, N-methyl pyrrolidone, 2-pyrrolidone, N-ethyl pyrrolidone, N-propyl pyrrolidone, N-formyl morpholine, dimethylsulfone, diethylsulfone, methylethylsulfone, dipropylsulfone, dibutylsulfone, tetraethylene glycol, triethylene glycol, dimethylene glycol, ethylene glycol, ethylene carbonate, propylene carbonate, and mixtures thereof. The presently preferred solvents are sulfolane, 3-methylsulfolane, N-formyl morpholine, 2-pyrrolidone, dipropylsulfone, tetraethylene glycol, and mixtures thereof.

In the process according to an embodiment of the invention, the extractive solvent includes a co-solvent. For example, a preferred solvent comprises sulfolane with 3-methylsulfolane, N-formyl morpholine, 2-pyrrolidone, dipropylsulfone, tetraethylene glycol, water, heavy sulfur residuals from FCC gasoline, or mixtures thereof as a co-solvent.

Feedstocks FCC gasoline contains many different types of sulfur species, including, without limitation, mercaptans, sulfides, disulfides, thiophenes, and benzothiophenes. Table 1 illustrates the commonly observed sulfur compounds that are extracted from hydrocarbon feedstocks using processes of the invention along with their normal boiling points.

TABLE 1

| Extractable Sulfur Compounds | nbp (° C.) |
|---|---|
| Ethyl methyl sulfide | 65-67 |
| Thiophene | 84.4 |
| Dimethyl disulfide (methyl disulfide) | 109.7 |
| 1-Methyl-1-propanethiol | 85 |
| Methyl mercaptan | 5.9 |
| Ethyl mercaptan | 35 |
| Dimethyl sulphide | 36.2 |
| Carbon disulphide | 46.2 |
| Iso-propyl mercaptan | 57-60 |
| Tert-butyl mercaptan | 64-64.2 |
| Methyl ethyl sulphide | 120.2 |
| Propyl mercaptan | 67 |
| Thiophene | 84.4 |
| Diethyl sulphide | 92 |
| Isobutyl mercaptan | 88 |
| Butyl mercaptan | 98 |
| Dimethyl disulphide | 109.7 |
| Diethyl disulphide | 152-154 |
| Diisopropyl disulphide | 175-176 |
| Dipropyl disulphide | 195-196 |
| Pentyl mercaptan | 127 |
| Hexyl mercaptan | 152 |
| Heptyl mercaptan | 176 |

In an alternate embodiment of the claimed invention, impurity and sulfur removal is carried out by using divided wall distillation in the solvent recovery process. This is shown in FIG. 3. A hydrocarbon feedstock is subjected to extractive distillation to extract the sulfur compounds and aromatics into an extract stream. At the same time, olefinic, naphthenic, and paraffinic compounds in the feedstock are rejected into a raffinate stream. The extract stream comprising the aromatics and sulfur compounds is subjected to a solvent recovery step using stripping stream. Here too, sulfur compounds and the impurities are concentrated and removed through a side draw. Recycled solvent from the solvent recovery column is subsequently introduced into the extractive distillation column and the upper portion of the solvent recovery column.

FIG. 4 shows an alternate method of removing sulfur and impurities by using additional solvent stripping. A hydrocarbon feedstock is subjected to an extractive process in a first extraction column (EDC1) to extract the sulfur compounds, impurities and aromatics into an extract stream. The extract stream from the first extraction column comprising the aromatics, impurities and sulfur compounds is subjected to a second extractive distillation step (EDC2) or a solvent recovery step (SRC1). In certain embodiments of the invention, a guard bed is used in conjunction with EDC2 or SRC1 to ensure removal of higher hydrocarbons and other impurities from the extracted aromatics hydrocarbons. In certain embodiments, an additional solvent stripping process is carried out in a second solvent recovery column wherein the extract of sulfur compounds is subjected to stripping by steam to produce a concentrated extract of impurities and sulfur compounds. Recycled solvent from the solvent recovery column is subsequently introduced into the extractive distillation column (EDC1 and EDC2) and the upper portion of the solvent recovery column.

FIG. 5 illustrates a further embodiment of the invention, which is a process for the removal of sulfur compounds and impurities using dual extraction units. In this process, a first extraction column is used to extract aromatics with sulfur compounds, which extract is treated in a second extraction column to separate aromatics from both the sulfur compounds and other impurities.

The claimed invention is an improvement to currently known processes. It provides an alternate/better method to obtain high purity extracted products by further separating the extracts into two streams, an overhead aromatic product and a side draw product that concentrates the sulfur compounds and impurities, which can then be much easily processed (by hydrotreating or other methods).

In accordance with an embodiment of the invention, benzene and toluene were extracted from a feedstock. The components of the raffinate and side draw were analyzed. The results of the analysis are shown below in Table 2.

TABLE 2

| | Boiling | Recovery |
|---|---|---|
| Wt % | Pt (° C.) | in Extract |
| Benzene | 80.1 | ~99% |
| Toluene | 110.6 | 99% |

All compounds below are recovered in the Side Draw

| | | |
|---|---|---|
| Mercaptans | | ~52% |
| Sulfides | | ~70% |
| Thiophene | 84.4 | ~99% |
| 2 Methyl Thiophene | | ~99% |
| 3 Methyl Thiophene | | ~99% |
| MEK | 79.6 | >95% |
| 2 Methyl 2 Butanone | | >95% |
| 2 pentanone | 101 | >95% |
| 3 pentanone | 102 | >95% |
| 1 Propanol | 97 | >95% |
| Tert., Sec & Iso Butanol | ~90 | >95% |
| 1 Butanol | 118 | >95% |
| Ethanol | 78 | >95% |
| Nitrile (Pyrrolidine, Propyl | | >95% |
| cyanide & Heavier) | | |

In the preceding detailed description, the invention is described with reference to specific exemplary embodiments thereof. Various modifications and changes may be made thereto without departing from the broader spirit and scope of the invention as set forth in the claims. The specification and drawings are, accordingly, to be regarded in an illustrative rather than a restrictive sense.

What is claimed is:

1. A method of separating impurities from hydrocarbons without the need for a hydrotreating step, the method comprising the step of providing a side draw in the second distillation column of a distillation system comprising a first and second distillation column.

2. The method of claim 1, wherein the extractive system is a divided wall system.

3. The method of claim 1, wherein the extractive system utilizes a column for additional solvent stripping.

4. The method of claim 1, wherein the extractive system utilizes an additional extraction unit.

5. The method according to claim 1 wherein said extractive process comprises distillation by extraction distillation or liquid-liquid distillation.

6. The method according to claim 5 wherein the extractive process is carried out using solvents that are selected from the group consisting of sulfolane, 3-methylsulfolane, 2,4-dimethylsulfolane, 3-ethylsulfolane, N-methyl pyrrolidone, 2-pyrrolidone, N-ethyl pyrrolidone, N-propyl pyrrolidone, N-formyl morpholine, dimethylsulfone, diethylsulfone, methylethylsulfone, dipropylsulfone, dibutylsulfone, tetraethylene glycol, triethylene glycol, dimethylene glycol, ethylene glycol, ethylene carbonate, propylene carbonate, and mixtures thereof.

7. The method according to claim 6, wherein the solvent comprises sulfolane combined with 3-methylsulfolane, N-formyl morpholine, 2-pyrrolidone, dipropylsulfone, tetraethylene glycol, water, heavy sulfur residuals from FCC gasoline, or mixtures thereof as a co-solvent.
